(19) United States
(12) Patent Application Publication
(10) Pub. No.: US 2023/0000930 A1
(43) Pub. Date: Jan. 5, 2023

(54) COMPOSITION FOR ELEVATING ABILITY OF BRAIN TISSUE AND USES THEREOF

(71) Applicant: GLAC BIOTECH CO., LTD., TAINAN CITY (TW)

(72) Inventors: HSIEH-HSUN HO, Tainan City (TW); YI WEI KUO, Tainan City (TW); KO-CHIANG HSIA, Tainan City (TW); WEN-YANG LIN, Tainan City (TW); JIA-HUNG LIN, Tainan City (TW); CHI-HUEI LIN, Tainan City (TW); CHENG-RUEI LIU, Tainan City (TW); SHU-HUI CHEN, Tainan City (TW)

(21) Appl. No.: 17/727,462

(22) Filed: Apr. 22, 2022

(30) Foreign Application Priority Data
Apr. 23, 2021 (TW) 110114810

(51) Int. Cl. A61K 35/747; A61K 9/00; A61K 35/745; A61P 25/28; C12N 1/20; A23L 33/135

(52) U.S. Cl. CPC A61K 35/747; A61K 9/0056; A61K 35/745; A61P 25/28; C12N 1/205; A23L 33/135; C12R 2001/225

(57) ABSTRACT

A composition, which comprises at least one of an AP-32 strain of Lactobacillus salivarius subsp. salicinius, a BLI-02 strain of Bifidobacterium longum subsp. infantis and fermentation metabolites thereof, has a physiological activity of elevating ability of brain tissue. The present invention may be used in form of a food composition or a pharmaceutical composition to elevate the ability of brain tissue.

### BACKGROUND OF THE INVENTION

### 1. Field of the Invention

The present invention relates to a composition and the applications thereof, particularly to a composition for elevating the ability of brain tissue, which contains at least one of lactic acid bacterium strains and the fermentation metabolites thereof, and the applications thereof.

### 2. Description of the Prior Art

According to US statistics, about 1% population aged over 60 years old suffer Parkinson's disease (PD). In Taiwan, data shows that the average age of onset of PD is about 62 years old and that the prevalence rate of PD is about 1%. The probability that males suffer PD is about 1.5 times the probability of females. The primary pathology of PD is the degeneration of the dopaminergic neurons of substantia nigra pars compacta of midbrain, which would affect the movement ability of patients progressively and leads to bradykinesia, tremor, stiffness, and posture instability.

The substantia nigra pars compacta of midbrain contains massive dopaminergic neurons, which transmit signals to basal ganglia. The cause why PD is accompanied by degeneration of dopaminergic neurons has not been settled yet so far. In a primary group (about 60-70%) of PD patients, the midbrain substantia nigra cells are degenerated by heredity, brain injury, chemical toxicity, etc. The degeneration of the midbrain substantia nigra cells leads to hypocrinia of dopamine (a neuro-transmitter) or inbalance of acetylcholine (Ach) and dopamine. The striatum of basal ganglia, which receives dopamine from substantia nigra as the neuro-transmitter, is thus affected and unable to regulate the signals of cerebral cortex, thalamus and the extrapyramidal system. Then, the function of coordinating muscles fails to work.

Many researches show that Parkinsonism is usually accompanied by variation of the metabolites of intestinal microbiota. Especially, the patients of Parkinsonism have a very high morbidity of Helicobacter pylori. Besides, the recent study found that Parkinsonism is accompanied by inbalance of bacteria microbiota, such as the decrease of Genuses Blautia, Coprococcus and Roseburia and the increase of Genus Ralstonia in fecal microbiota.

Some researches show that certain lactic acid bacterium strains not only can treat diseases but also can relieve the problems of cognitive functions and emotional damage. For example, Dinan et al. expanded the concept of probiotics and proposed psychobiotics. The concept of psychobiotics is that taking appropriate amount of active microorganisms is favorable to the health of psychiatric patients, such as enhancing the disease resistance of intestinal tracts, improving the bacteria flora of intestinal tracts, regulating immunological systems, and helping digestion (Dinan et al., (2013). “Psychobiotics: a novel class of psychotropic”. Biological Psychiatry, 74(10):720-6. doi: https://doi.org/10.1016/j.biopsych.2013_05.001). It is found recently: feeding lactic acid bacteria may influence the behavior of hosts and the function of brains. For example, Desbonnet et al. found that feeding Bifidobacterium infantis 35624 may improve depressive disorder of mice and increase neuro-transmitters in the brains of mice (Desbonnet et al., (2008). “The probiotic Bifidobacteria infantis: An assessment of potential antidepressant properties in the rat”. Journal of Psychiatric Research, 43(2): 164-174. doi: https://doi.org/10.1016/j.jpsychires.2008.03.009).

In general, lactic acid bacteria are safe to human bodies and favorable to health. It has been a target the manufacturers are eager to achieve: developing nutritional supplement products safe to human bodies and suitable to use persistently to elevate the ability of brain tissue.

### SUMMARY OF THE INVENTION

The present invention provides a composition containing at least one of lactic acid bacterium strains and the fermentation metabolites thereof, which has a physiologically-active effect of elevating the ability of brain tissue, whereby the composition containing at least one of lactic acid bacterium strains and the fermentation metabolites thereof of the present invention may be used to elevate the ability of brain tissue and may be fabricated in form of a food composition or a pharmaceutical composition.

In one embodiment, the present invention proposes an application of using at least one of lactic acid bacterium strains and the fermentation metabolites thereof to fabricate a food composition for elevating the ability of brain tissue, wherein the food composition comprises at least one of isolated lactic acid bacterium strains and the fermentation metabolites thereof and a physiologically-acceptable recipient, diluent or carrier. The isolated lactic acid bacterium strain is selected from a group including an AP-32 strain of Lactobacillus salivarius subsp. salicinius (CCTCC NO: M2011127); a BLI-02 strain of Bifidobacterium longum subsp. infantis (CGMCC No. 15212); or combinations thereof. The abovementioned strains are respectively deposited in China Center for Type Culture Collection (CCTCC) and China General Microbiological Culture Collection Center (CGMCC).

In one embodiment, the present invention proposes an application of using at least one of lactic acid bacterium strains and the fermentation metabolites thereof to fabricate a pharmaceutical composition for elevating the ability of brain tissue, wherein the pharmaceutical composition comprises at least one of isolated lactic acid bacterium strains and the fermentation metabolites thereof and a pharmaceutically-acceptable excipient, diluent or carrier. The isolated lactic acid bacterium strain is selected from a group including an AP-32 strain of Lactobacillus salivarius subsp. salicinius (CCTCC NO: M2011127); a BLI-02 strain of Bifidobacterium longum subsp. infantis (CGMCC No. 15212); or combinations thereof. The abovementioned strains are respectively deposited in China Center for Type Culture Collection (CCTCC) and China General Microbiological Culture Collection Center (CGMCC).

In one embodiment, the present invention proposes a composition containing at least one of lactic acid bacterium strains and the fermentation metabolites thereof, which comprises at least one of isolated lactic acid bacterium strains and the fermentation metabolites thereof and a physiologically or pharmaceutically-acceptable recipient, diluent or carrier. The isolated lactic acid bacterium strain has a physiologically-active effect of elevating the ability of brain tissue. The isolated lactic acid bacterium strain is selected from a group including an AP-32 strain of Lactobacillus salivarius subsp. salicinius (CCTCC NO: M2011127); a BLI-02 strain of Bifidobacterium longum subsp. infantis (CGMCC No. 15212); or combinations thereof. The abovementioned strains are respectively deposited in China Center for Type Culture Collection (CCTCC) and China General Microbiological Culture Collection Center (CGMCC).

The objective, technologies, features and advantages of the present invention will become apparent from the following description in conjunction with the accompanying drawings wherein certain embodiments of the present invention are set forth by way of illustration and example.

### BRIEF DESCRIPTION OF THE DRAWINGS

The foregoing conceptions and their accompanying advantages of this invention will become more readily appreciated after being better understood by referring to the following detailed description, in conjunction with the accompanying drawings, wherein:

FIG. 1 shows the average variation of body weights of each group of rats;

FIG. 2 shows the experiment results of the influence of the disease-inducing operation by 6-OHDA on the body rotation numbers of the rats per minute;

FIG. 3 shows the experiment results of the influence of the disease-inducing operation by 6-OHDA on the time that the rat stays in a balance bar for evaluating the synergy and balance of the rats;

FIG. 4 shows the experiment results of TH+ levels of the striatum of the rats, wherein the TH+ level of the brain tissue of the non-pathological side is used as a standard (100%) to normalize the TH+ level of the brain tissue of the pathological side;

FIG. 5 shows the experiment results of TH+ levels of the substantia nigra pars compacta of the rats, wherein the TH+ level of the brain tissue of the non-pathological side is used as a standard (100%) to normalize the TH+ level of the brain tissue of the pathological side;

FIG. 6 shows the results of the analysis of the basal oxygen consumption rate (OCR) of mitochondria in the brain tissues of the rats, wherein the result of the Group ND is used as a standard (100%) to normalize the results of the other groups;

FIG. 7 shows the results of the analysis of the basal oxygen consumption rate (OCR) of mitochondria in the muscle tissues of the rats, wherein the result of the Group ND is used as a standard (100%) to normalize the results of the other groups;

FIG. 8 shows the experiment results of the effects of single lactic acid bacterium strains on protecting nerve cells, wherein the result is expressed by the survival rate of nerve cells (%);

FIG. 9 shows the experiment results of the nerve cell protection effects of the mixtures of lactic acid bacterium strains of the present invention by different ratios, wherein the result is expressed by the survival rate of nerve cells (%); and

FIG. 10 shows the experiment results of the nerve cell protection effects of the mixtures where the lactic acid bacterium strain of the present invention is mixed with a different strain of the same genus and the same species by a ratio of 1:1, wherein the result is expressed by the survival rate of nerve cells (%).

### DESCRIPTION OF THE PREFERRED EMBODIMENTS

Various embodiments of the present invention will be described in detail below and illustrated in conjunction with the accompanying drawings. In addition to these detailed descriptions, the present invention can be widely implemented in other embodiments, and apparent alternations, modifications and equivalent changes of any mentioned embodiments are all included within the scope of the present invention and based on the scope of the Claims. In the descriptions of the specification, in order to make readers have a more complete understanding about the present invention, many specific details are provided; however, the present invention may be implemented without parts of or all the specific details. In addition, the well-known steps or elements are not described in detail, in order to avoid unnecessary limitations to the present invention. Same or similar elements in Figures will be indicated by same or similar reference numbers. It is noted that the Figures are schematic and may not represent the actual size or number of the elements. For clearness of the Figures, some details may not be fully depicted.

The freeze-dried cultures of the lactic acid bacterium strains mentioned in the specification are deposited in China Center for Type Culture Collection (CCTCC) of Wuhan University (Wuhan 430072, China) and China General Microbiological Culture Collection Center (CGMCC) of Chinese Academy of Sciences (NO. 1 West Beichen Road, Chaoyang District, Beijing 100101, China)). The details thereof are listed in Table.1.

TABLE 1
Data of Deposited Lactic Acid Bacterium Strains

| Strain | Specie | Deposition No. | Deposition Date |
|---|---|---|---|
| AP-32 | Lactobacillus salivarius | CCTCC NO: | Apr. 10, 2011 |
| | subsp. salicinius | M2011127 | |
| BLI-02 | Bifidobacterium longum | CGMCC No. | Jan. 15, 2018 |
| | subsp. infantis | 15212 | |

It is found: the AP-32 strain of Lactobacillus salivarius subsp. salicinius (CCTCC NO: M2011127) and the BLI-02 strain of Bifidobacterium longum subsp. infantis (CGMCC No. 15212) listed in Table.1 have a physiologically-active effect of elevating the ability of repairing brain tissue. Therefore, the AP-32 strain of Lactobacillus salivarius subsp. salicinius and the BLI-02 strain of Bifidobacterium longum subsp. infantis listed in Table.1 and the fermentation metabolites thereof may be used to elevate the ability of repairing brain tissue.

In one embodiment, the composition for elevating the ability of repairing brain tissue of the present invention comprises the isolated AP-32 strain of Lactobacillus salivarius subsp. salicinius, the isolated BLI-02 strain of Bifidobacterium longum subsp. infantis or the fermentation metabolites thereof, and a recipient, diluent or carrier. The Deposition No. of the AP-32 strain of Lactobacillus salivarius subsp. salicinius is CCTCC NO: M2011127. The Deposition No. of the BLI-02 strain of Bifidobacterium longum subsp. infantis is CGMCC No. 15212. They are respectively deposited in China Center for Type Culture Collection (CCTCC) and China General Microbiological Culture Collection Center (CGMCC).

In one embodiment, the excipient diluent or carrier may be a physiologically-acceptable excipient, diluent or carrier, whereby the composition of the present invention may be used as a food composition. In one embodiment, the excipient, diluent or carrier may be a pharmaceutically-acceptable excipient, diluent or carrier, whereby the composition of the present invention may be used as a pharmaceutical composition.

In the embodiment of a food composition, the physiologically-acceptable excipient, diluent or carrier may be a food. The food may be but is not limited to be dairy food, tea, coffee, candy (such as an oral strip, a chewable tablet, or jelly sweets), functional beverages or a combination thereof. The dairy food may be fermented milk, yoghurt, cheese or powdered milk.

In the embodiment of a pharmaceutical composition, the pharmaceutical composition may be in form of an oral dosage. For example, the oral dosage may be in form of a tablet, a capsule, a solution, or a powder.

In the embodiment of a food composition or a pharmaceutical composition, the AP-32 strain of Lactobacillus salivarius subsp. salicinius is an active strain, and the number of the AP-32 strain of Lactobacillus salivarius subsp. salicinius may be over 10⁶ CFU (Colony-Forming Unit), preferably over 10¹⁰ CFU. In one embodiment, the AP-32 strain of Lactobacillus salivarius subsp. salicinius is a deactivated strain. In the embodiment of the composition containing a fermentation metabolite of a lactic acid bacterium, the fermentation metabolite may be a fermentation liquid containing a deactivated strain, a fermentation liquid where bacteria are removed, or a dried powder of the abovementioned fermentation liquid. In one embodiment, the fermentation liquid is a supernatant of fermentation, or a whey of fermentation. In one embodiment, the fermentation metabolite of a lactic acid bacterium contains more than 0.5 wt. % of the powder of the fermentation metabolite or more than 2.5 wt. % of the fermentation liquid of the fermentation metabolite.

### Embodiment I: Morphology and General Properties of the Strains of the Present Invention

The taxonomic characteristics of the strain are identified with the 16S rDNA sequencing analysis and the API bacterial identification system. The morphology and general properties of the strains are listed in Table.2.

TABLE 2
Morphology and General Properties of Lactic Acid
Bacterium Strain of the Present Invention

| Strain | Morphology and characteristics |
|---|---|
| AP-32 strain of | 1.They are gram-positive bacilli, unlikely to |
| Lactobacillus | generate spores, free of catalase, oxidase and |
| salivarius subsp. | motility, able to grow in aerobic and anaerobic |
| salicinius | environments, most suitable to grow at a |
| | temperature of 37 ± 1° C. They belong |
| | to facultative heterofermentative strains and |
| | do not generate gas in glucose metabolism. |
| | 2. The colonies grown in MRS agar are in form |
| | of solid circles in white color. The bodies of |
| | the bacteria each have a shape of a short rod, |
| | and the ends of the body are circular-shaped. |
| | They often appear in single bodies. |
| BLI-02 strain of | 1.They are gram-positive bacilli, unlikely to |
| Bifidobacterium | generate spores, free of catalase, oxidase and |
| longum subsp. | motility, able to grow in obligately-anaerobic |
| infantis | environments, most suitable to grow at a |
| | temperature of 37 ± 1° C. They belong to |
| | facultative heterofermentative strains and do |
| | not generate gas in glucose metabolism. |
| | 2. The colonies grown in MRS agar are in form of |
| | solid circles in white color. The bacterium body |
| | has a middle-size or longer rod-like shape, and |
| | two ends thereof sometimes have Y or V-shaped |
| | branches. |

### Embodiment II. Collection, Cultivation and Preservation of the Lactic Acid Bacterium Strains of the Present Invention

The strain of the present invention is preserved in 20% glycerol at a temperature of −80° C. Before use, the strain is activated twice with MRS broth (DIFCO) containing 0.05% cysteine at a temperature of 37° C. for 24 hours. The AP-32 strain of Lactobacillus salivarius subsp. Salicinius is sourced from human intestinal tracts. The BLI-02 strain of Bifidobacterium longum subsp. infant is sourced from healthy human breast milk. In one embodiment, the liquid culture medium includes carbon sources and nitrogen sources. For example, the carbon source may be glucose, fructose, lactose, sucrose, maltose, galactose, mannose, trehalose, starch, molasses, potato starch, maize starch, malt extract, maltodextrin, or a combination thereof. The nitrogen source may be (NH₄)₂SO₄, (NH₄)₃PO₄, NH₄NO₃, NH₄Cl, casamino acid, urea, peptone, polypeptone, tryptone, meat extract, yeast extract, yeast powder, milk, soybean flour, whey, or a combination thereof. In one embodiment, the liquid culture medium comprises 2-5 wt. % of a mixture of glucose and maltodextrin, preferably 3 wt. % of a mixture of glucose and maltodextrin. In one embodiment, the liquid culture medium comprises at least one of 5-30 wt. % milk and 1-10 wt. % soybean flour.

The fermentation metabolite is the fermentation product generated by the fermentation of the lactic acid bacterium strains of the present invention in the liquid culture medium. The fermentation product is centrifuged, filtered, sterilized and then purified to obtain a fermentation liquid. According to requirement, the fermentation liquid is further dried to form fermentation powder of the lactic acid bacterium. The fermentation powder or the aqueous solution of the fermentation powder can be stored at an ambient temperature.

### Embodiment III: Animal Disease-Inducing Mode of Parkinsonian Rats

The experiment uses 6-Hydroxydopamine (6-OHDA) to induce rats to suffer parkinsonism-like syndromes. The animal disease-inducing mode used by the experiment injects 6-OHDA to rats unilaterally to damage the unilateral dopaminergic system and induce the hemi-parkinsonism. The unilateral injection mode is often adopted at present because it can overcome the difficult of nursing the rats receiving bilateral injection of 6-OHDA. A stereotaxic instrument and a microinjector are used to inject 6-OHDA into a region between the striatum and the substantia nigra pars compacta to damage the dopaminergic neurons of the substantia nigra pars compacta and decrease the dopamine released to the striatum, whereby to induce rats to suffer parkinsonism-like diseases.

Unilateral 6-OHDA disease-inducing process: Mix Tiletamine and Zolazepam (TZ) by a ratio of 1:1 to form an intramuscular injection anesthetics. Use the abovementioned anesthetics (2.5 mg/kg) and ROMPUN (0.3 mg/kg) to anesthetize all the rats having been disease-induced by 6-OHDA. Before being injured by 6-OHDA, Norpramin (25 mg/kg) is intraperitoneally into all the rats to protect the arterenol neurons thereof. Use a micro drill to drill a hole on the right frontal bone. Dissolve 12 g 6-OHDA (purchased from Sigma) in 4 L of an aseptic solution containing 0.9% NaCl and 0.02% ascorbic acid. Use a Hamilton microliter syringe to inject the abovementioned 6-OHDA solution into the medial forebrain bundle (MFB) at a flowrate of 0.5μL/min. According to the stereotaxic atlas of Paxinos and Watson, the stereotaxic coordinates of the injected position are 4.3 mm at the rear of the epistomal suture, 1.3 mm from the lateral of the epistomal suture and 7.8 mm under the dura mater. The 6-OHDA solution is injected into the injected position at a flowrate of 0.5 μL/min. Less than 10 minutes later, withdraw the syringe slowly, and let the rats recover from narcosis.

In order to evaluate the effect of unilateral 6-OHDA injection, perform intraperitoneal injection of Apomorphine. Apomorphine will increase the dopaminergic post-synaptic receptor of the striatum. In the situation of dopamine decrease, the dopamine transference is weakened on the corresponding side, and the rats rotate because of inbalance. While a rat rotates more than 100 circles per hour, the rat is confirmed to have Parkinson's disease.

The rats used in the experiment are Sprague Dawley (SD) male rats purchased from BioLASCO Taiwan Co., Ltd., weighing 300 g and 6-8 weeks old. The rats are accommodated in terraria at a temperature of 22±2° C. and a humidity of 55±5% with a light cycle and a dark cycle respectively of 12 hours. Feed the rats with the MFG solid fodder, which is purchased from BioLASCO Taiwan Co., Ltd., for an adaption period of a week. The general solid fodder used to feed control group during the experiment and the tested animals during one-week adaption period is the MFG fodder having a calorific value of 3.57 kcal/g. The rats are allowed to take animal fodder (Chow 5001) and water freely. Thirty 6-8 weeks old Sprague Dawley rats are used in the experiment. Each five rats are randomly assigned to be one of the following groups:

(1) Normal diet control group (Group ND): the rats are neither injected with 6-OHDA nor fed with the lactic acid bacterium strains or the fermentation metabolites thereof;
(2) PD-induced group (Group PD): the rats are injected with 6-OHDA; 3 weeks and 6 weeks later, apomorphine is supplied to the rats to evaluate the Parkinsonism-inducing effect;
(3) L-dopa therapy group (Group LD): the rats have been injected with 6-OHDA, and the disease-inducing effect has been verified; the rats are continuously tube-fed with L-dopa by a dosage of 6 mg/kg for 14 weeks;
(4) Experiment Group I (Group AP-32): the rats have been injected with 6-OHDA, and the disease-inducing effect has been verified; the rats are continuously tube-fed with the AP-32 strain of Lactobacillus salivarius subsp. salicinius of the present invention by a one-fold dosage of 1.03×10⁹ CFU/kg/day for 14 weeks;
(5) Experiment Group II (Group BLI-02): the rats have been injected with 6-OHDA, and the disease-inducing effect has been verified; the rats are continuously tube-fed with the BLI-02 strain of Bifidobacterium longum subsp. infantis of the present invention by a one-fold dosage of 1.03×10⁹ CFU/kg/day for 14 weeks;
(6) Experiment Group III (Group gL-57): the rats have been injected with 6-OHDA, and the disease-inducing effect has been verified; the rats are continuously tube-fed with the gL-57 strain of Bifidobacterium breve by a one-fold dosage of 1.03×10⁹ CFU/kg/day for 14 weeks;
(7) Experiment Group IV (Group gL-39): the rats have been injected with 6-OHDA, and the disease-inducing effect has been verified; the rats are continuously tube-fed with the gL-39 strain of Bifidobacterium animalis by a one-fold dosage of 1.03×10⁹ CFU/kg/day for 14 weeks;
(8) Experiment Group V (Group AP-32MR): the rats have been injected with 6-OHDA, and the disease-inducing effect has been verified; the rats are continuously tube-fed with the fermentation metabolite of the AP-32 strain of Lactobacillus salivarius subsp. salicinius of the present invention by a dosage of 62 mg/kg/day for 14 weeks;
(9) Experiment Group VI (Group BLI-02MR): the rats have been injected with 6-OHDA, and the disease-inducing effect has been verified; the rats are continuously tube-fed with the fermentation metabolite of the BLI-02 strain of Bifidobacterium longum subsp. infantis of the present invention by a dosage of 62 mg/kg/day for 14 weeks.

Referring to the dosage of lactic acid bacterium supplement recorded in documents and according to the recommended daily intake of human body, the recommended daily dosage of lactic acid bacteria supplement for each person is about 1×10¹⁰ CFU. One-fold of the abovementioned dosage is used in the experiment. For a person weighing 60 kg, the recommended daily intake of lactic acid bacteria is 1×10¹⁰ CFU, and the recommended daily intake of the fermentation metabolites of lactic acid bacteria is 600 mg. The metabolic rate of human being is different from the metabolic rate of a different animal. According to the method of “Estimating the maximum safe starting dose in initial clinical trials for therapeutics in adult healthy volunteers (US FDA, 2005)”, the dosage for human body is converted into the dosage for the tested animal. The conversion coefficient of the rat to the human being is 6.2. Therefore, one-fold dosage of lactic acid bacteria for the rat is 10¹⁰ CFU/60 kg×6.2=1.03×10⁹ CFU/kg; one-fold dosage of the fermentation metabolites of lactic acid bacteria for the rat is 600 mg/60 kg×6.2=62 mg/kg. The individual dosage of the tested sample for each rat is calculated according to the weight of each rat. The dosage of the tested sample is dissolved in 1 ml deionized water. The solution is tube-fed to the rat directly, and the MFG solid fodder is supplied to the rat simultaneously. The groups, which are not tube-fed with the lactic acid bacteria or the metabolites thereof, are tube-fed with the same volume of deionized water.

### Embodiment IV: Analysis of SCFA and MCFA in Rats' Excrement

The present invention uses the gas chromatography-mass spectrometry (GC-MS) method to analyze the contents of the short chain fatty acid (SCFA) and the middle chain fatty acid (MCFA) in the rats' excrement. The analysis includes steps:

(1) Collect and freeze-dry the contents of the intestinal tracts and samples of the excrement of the rats, and grind it into powder;
(2) Add 150 mg frozen dry powder into 150 μL 1M hydrochloric acid saturated with sodium chloride; then add to the solution 1.5 mL ethyl acetate containing an internal standard 2-ethyl butyric acid (the concentration of the internal standard is 5 μg/mL);
(3) Place the sample in a homogenizer and grind and mix it uniformly; then centrifuge the sample at a speed of 10000 rpm for 10 minutes;
(4) Take 220 μL supernatant of the sample, and dry the supernatant with 100 mg magnesium sulfate; then centrifuge it at a speed of 18000 rpm for 10 minutes;
(5) Take 150 μL supernatant of the sample, and dry the supernatant with 50 mg magnesium sulfate; next centrifuge it at a speed of 18000 rpm for 10 minutes; then, seal 72 μL supernatant in an airtight bottle;
(6) Add 18 μL derivatization reagent MTBSTFA into the bottle and screw the bottle cap tightly; mix the liquid evenly and place the bottle in a water bath at a temperature of 80° C. for 20 minutes;
(7) Place the sample at an ambient temperature for 8 hours for derivatization, and use the GC-MS method to perform analysis; take 1 μL of the derivatized sample and use an automatic sampler to inject the derivatized sample into the instrument for analysis.

The gas chromatography-mass spectrometry kit is a combination of Thermo Finnigan Trace GC 2000, Polaris Q mass detector and the mass spectrometry software Xcalibur. The chromatographic column is the Rtx-5MS capillary column (Restek) having dimensions of 30 m×0.25 mm×0.25 μm. The temperatures of the injector and the ion source are respectively 230° C. and 200° C. According to different retention times and mass-spectrometry characteristics, the peaks detected in the GC-MS analysis are used to identify the composition and quantities of the intermediate metabolites involved with energy metabolism through the comparison with the market-available standard samples and the searches in the NIST and Wiley database, whereby to understand the preference of energy metabolism.

The results of the analysis of the contents of the short chain fatty acid (SCFA) and the middle chain fatty acid (MCFA) in the rats' excrement are shown in Table.1, wherein the symbol a indicates that the result has significant difference with respect to Group ND, and p<0.05; the symbol b indicates that the result has significant difference with respect to Group PD, and p<0.05. Table.1 shows that among four tested lactic acid bacterium strains, the AP-32 strain of Lactobacillus salivarius subsp. salicinius of and the BLI-02 strain of Bifidobacterium longum subsp. infantis the present invention have better output of the short chain fatty acid (SCFA), especially the output of acetic acid. The contents of propionic acid, isobutyric acid, butyric acid, isovaleric acid, hexanoic acid, and heptanoic acid in the intestinal tracts of the rats of Group AP-32 are obviously higher than those in Group PD. The contents of propionic acid, butyric acid, and isovaleric acid in the intestinal tracts of the rats of Group AP-32MR are obviously higher than those in Group PD. The contents of propionic acid, isobutyric acid, butyric acid, isovaleric acid, hexanoic acid, and heptanoic acid in the intestinal tracts of the rats of Group BLI-02 are obviously higher than those in Group PD. The contents of propionic acid, butyric acid, and isovaleric acid in the intestinal tracts of the rats of Group BLI-02MR are obviously higher than those in Group PD.

### Embodiment V: Analysis of the Average Weight and Body Composition of the Rats

The body compositions of the rates are detected with a body composition analyzer (DEXA, GE Lumar, Madison, Wis., USA). The rats fast for 8 hours. Next, each rat is anesthetized and fixed on a board with the four limbs spread. The information of the rats is input to the computer, such as birthday days, body lengths and weights. Then, start the measurements. Two different grades of X-rays are used to scan the bodies of the rats. Bones and soft tissues will respectively absorb different amounts of X-ray. The muscle and fat of the soft tissues will also respectively absorb different amount of X-ray. The muscle mass and the body fat ratio are worked out with the equation built in the instrument. The statistical analysis uses One-way ANOVA, tested with Tukey HSD, and expressed as mean±SEM.

The results of the analysis of the body composition of the rats are shown in Table.2, wherein the symbol a indicates that the result has significant difference with respect to Group ND, and p<0.05; the symbol b indicates that the result has significant difference with respect to Group PD, and p<0.05. Table.2 shows that the groups fed with the AP-32 strain of Lactobacillus salivarius subsp. salicinius and the BLI-02 strain of Bifidobacterium longum subsp. infantis of the present invention have higher total body fat, higher total muscle mass and higher bone density and that significant difference exists between Group PD and Group AP-32/Group BLI-02. Group AP-32 and Group BLI-02 also have better performance with respect to Group LD.

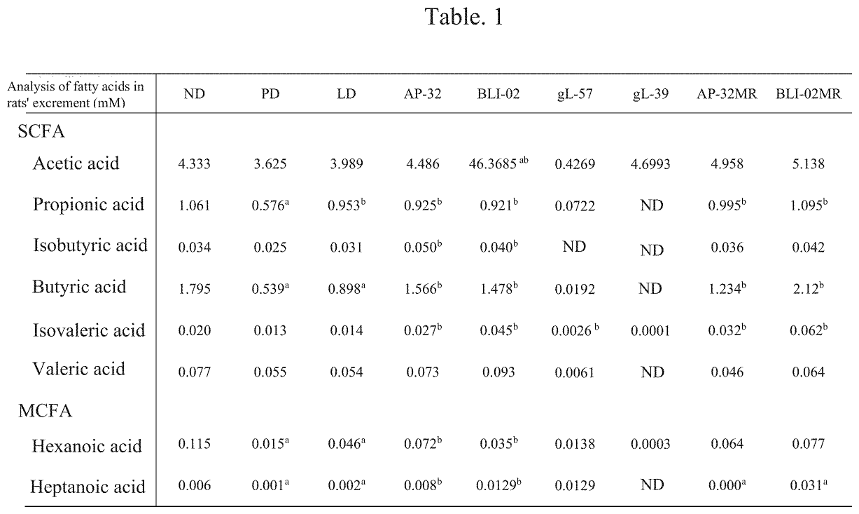

FIG. 1 shows the variation of the average body weight of each group of rats. FIG. 1 shows the disease-inducing operation of 6-OHDA injection causes muscle asynergy and affects food intake of the rats. Thus, the body weights of the rats decrease week by week. In Group LD, L-dopa therapy is ineffective in increasing the body weights of the rats. In Group AP-32 whose rats are fed with the AP-32 strain of Lactobacillus salivarius subsp. salicinius and Group BLI-02 whose rats are fed with the BLI-02 strain of Bifidobacterium longum subsp. infantis, the body weights of the rats increase gradually and approach the body weights of Group ND in the fourteenth week.

### Embodiment VI: The Evaluation of the Motor Functions of the Rats

The experiment uses the Rotarod to test the synergy and balance of the rats. The treadmill of the RT series is used to study the effect of medicine on the synergy and anti-fatigue ability of animals. The Rotarod treadmill is an ideal instrument to screen and identify anti-fatigue drugs. The instrument is suitable to perform some tests on rats and mice, such as fatigue tests, skeletal muscle relaxation tests, central nerve inhibition tests and other tests needing exercise to evaluate the effects of drugs. For example, the instrument may be used to test the influence of toxicity on motor ability, the influence of deficiency of some material on motor ability, and the influence of cerebro-cardiovascular drugs on motor ability. The statistical analysis uses One-way ANOVA, tested with Tukey HSD, and expressed as mean±SEM.

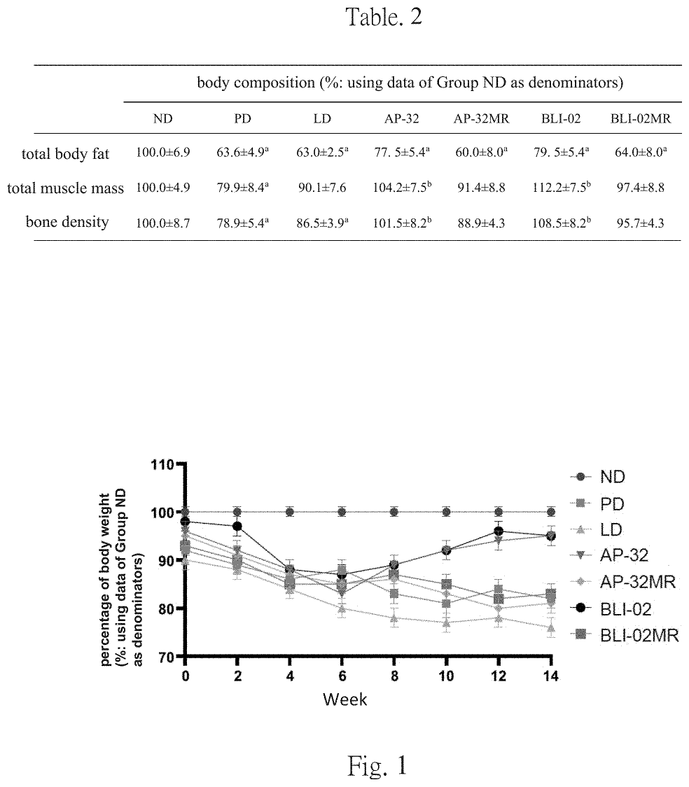

The disease-inducing operation by 6-OHDA unbalances the body of the rat and increases the number of body rotation per minute. Therefore, the body rotation number per minute may be used to estimate the influence of the disease-inducing operation by 6-OHDA on the rats. The experiment results of the influence of the disease-inducing operation by 6-OHDA on the body rotation numbers of the rats per minute are shown in FIG. 2, wherein the symbol a indicates that the result has significant difference with respect to Group ND, and p<0.05; the symbol b indicates that the result has significant difference with respect to Group PD, and p<0.05. It is learned from FIG. 2: Group LD whose rats are fed with L-dopa, Group AP-32 whose rats are fed with the AP-32 strain of Lactobacillus salivarius subsp. salicinius, Group BLI-02 whose rats are fed with the BLI-02 strain of Bifidobacterium longum subsp. infantis, Group AP-32MR whose rats are fed with the fermentation metabolites of the AP-32 strain of Lactobacillus salivarius subsp. salicinius and Group BLI-02MR whose rats are fed with the fermentation metabolites of the BLI-02 strain of Bifidobacterium longum subsp. infantis have obviously lower body rotation numbers than Group PD. It should be noted: Group AP-32 whose rats are fed with the AP-32 strain of Lactobacillus salivarius subsp. salicinius, Group BLI-02 whose rats are fed with the BLI-02 strain of Bifidobacterium longum subsp. infantis, Group AP-32MR whose rats are fed with the fermentation metabolites of the AP-32 strain of Lactobacillus salivarius subsp. salicinius and Group BLI-02MR whose rats are fed with the fermentation metabolites of the BLI-02 strain of Bifidobacterium longum subsp. infantis have better therapy effect than Group LD whose rats are fed with L-dopa.

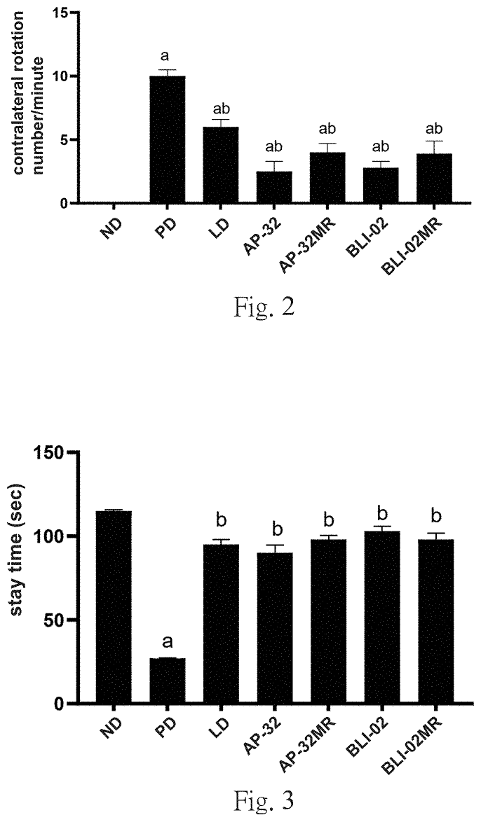

The disease-inducing operation by 6-OHDA damages the synergy and balance of the rat body. Therefore, the time that the rat stays in a balance bar may be used to estimate the influence of the disease-inducing operation by 6-OHDA on the rats. The experiment results of the influence of the disease-inducing operation by 6-OHDA on the synergy and balance of the rats are shown in FIG. 3, wherein the symbol a indicates that the result has significant difference with respect to Group ND, and p<0.05; the symbol b indicates that the result has significant difference with respect to Group PD, and p<0.05. It is learned from FIG. 3: Group LD whose rats are fed with L-dopa, Group AP-32 whose rats are fed with the AP-32 strain of Lactobacillus salivarius subsp. salicinius, Group BLI-02 whose rats are fed with the BLI-02 strain of Bifidobacterium longum subsp. infantis, Group AP-32MR whose rats are fed with the fermentation metabolites of the AP-32 strain of Lactobacillus salivarius subsp. salicinius and Group BLI-02MR whose rats are fed with the fermentation metabolites of the BLI-02 strain of Bifidobacterium longum subsp. infantis have obviously better balance and stay in a balance bar for a longer time than Group PD.

### Embodiment VII: Analysis of Tyrosine Hydroxylase Positive Level (TH+ Level) in the Brains of Rats

Dopamine is a member of the catecholamine group, functioning as a neuro-transmitter. Dopamine is also a hormone generated by the endocrine system, performing physiological functions, such as vasoconstriction regulation and glycogenolysis. Dopamine is a biogenic amine, derived from tyrosine through a series of reactions. The metabolic path of dopamine includes the following reactions: phenylalanine hydroxylase catalyzes phenylalanine to form tyrosine; tyrosine hydroxylase (TH) catalyzes tyrosine to form L-dopa; aromatic amino acid decarboxylase (AADC) or DOPA decarboxylase strips off one carbon dioxide molecule of L-dopa to form dopamine. Therefore, tyrosine hydroxylase (TH) plays an important role in the synthesis of brain dopamine.

In clinics, lacking tyrosine hydroxylase (TH) will interrupt the transference of neuro-transmitters and lead to deficiency of dopamine, norepinephrine, epinephrine and serotonin in the central nervous system and peripheral nervous system, wherein dopamine, norepinephrine and epinephrine are totally referred to as catecholamine. The phenomenon of lacking tyrosine hydroxylase is called tyrosine hydroxylase (TH) deficiency in clinical medicine. The expressions of TH deficiency vary greatly, including the mild TH-deficient dopa-responsive dystonia (DRD), the severe TH-deficient infantile Parkinsonism, and the further severer TH-deficient progressive infantile encephalopathy. The patients of mild symptoms are likely to fall ill in their childhoods. At beginning, the symptom may be unilateral dystonia, asymmetric four-limb dystonia, postural tremor, uncoordinated pace, etc. With progression of the disease, the patient may suffer classic dopa-responsive dystonic gait disorder. Pharmacotherapy is normally effective to the patients of mild symptoms. The patients of moderate symptoms may be unable or hard to walk. The therapy of the moderate-symptom patients is more difficult, needing cooperation of several drugs. However, the side-effects of the drugs may cause hyperactivity, irascibility, etc. Besides, the patients of moderate symptoms need long-term therapy.

In the experiment, after the rats are sacrificed, the skulls thereof are opened with a bone cutting tool to reveal the full brain tissues. A spatula is carefully moved along the peripheral of the brain tissue to take out the brain tissue. The brain tissue is placed in a 1.5 mL micro centrifuge tube and kept in a refrigerator at a temperature of −80° C. for experiments in the future. The cryogenic brain tissue section is treated with a primary antibody, which is selected from a group including the antiphospho-α-synucleinSer129 polyclonal antibody, the monoclonal anti-α-synuclein antibody, the anti-NeuN monoclonal antibody and the monoclonal anti-tyrosine hydroxylase antibody. Next, wash off the primary antibody and treat the section with a secondary antibody and streptavidin peroxidase conjugates. Next, stain the brain tissue section with 3′, 3′-diaminobenzendine (DAB), and use Stereo Investigator software (MBF Biosciences) to count NeuN+-neurons or TH+-dopaminergic neurons. Next, use ImageJ software (National Institutes of Health) to quantitate the staining result of TH+-dopaminergic neurons in the basal ganglia. Next, the TH+ level of the brain tissue of the non-pathological side is used as a standard (100%) to normalize the TH+ level of the brain tissue of the pathological side. The statistical analysis uses One-way ANOVA, tested with Tukey HSD, and expressed as mean±SEM.

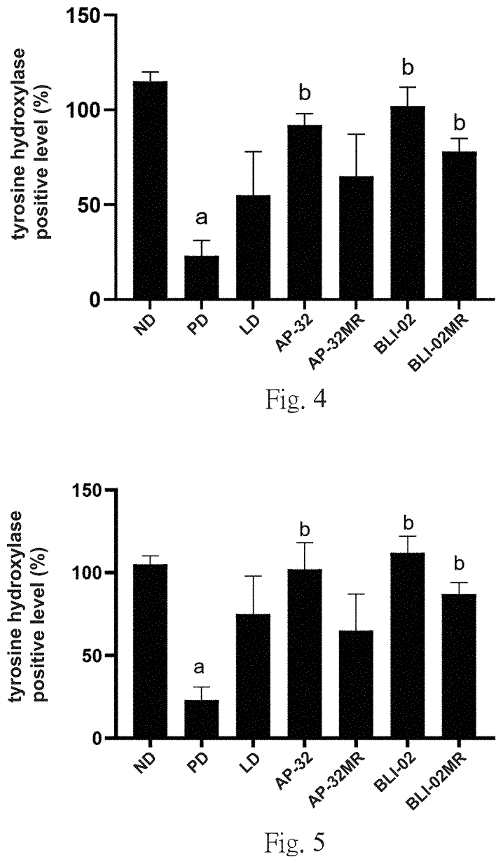

TH+ level in the brain tissue is used to evaluate the dopaminergic neurons after 6-OHDA is used to induce Parkinsonism of the rats. Unilaterally inject 6-OHDA, which will damage the dopaminergic neurons of the striatum and the substantia nigra pars compacta, into medial forebrain bundle (MFB). The experiment results of TH+ levels of the striatum are shown in FIG. 4, and the experiment results of TH+ levels of the substantia nigra pars compacta are shown in FIG. 5, wherein the symbol a indicates that the result has significant difference with respect to Group ND, and p<0.05; the symbol b indicates that the result has significant difference with respect to Group PD, and p<0.05. In the striatum and the substantia nigra pars compacta, the TH+ levels of Group PD indicate that the dopaminergic neurons of Group PD are obviously less than those of Group ND, as shown in FIG. 4 and FIG. 5. Group AP-32 whose rats are fed with the AP-32 strain of Lactobacillus salivarius subsp. salicinius, Group BLI-02 whose rats are fed with the BLI-02 strain of Bifidobacterium longum subsp. infantis, and Group BLI-02MR whose rats are fed with the fermentation metabolites of the BLI-02 strain of Bifidobacterium longum subsp. infantis have obviously increased TH+ levels in the striatum and the substantia nigra pars compacta and have significant statistical difference with respect to Group PD, as shown in FIG. 4 and FIG. 5.

From the experiment results mentioned above, it is learned: the lactic acid bacteria or the fermentation metabolites thereof can effectively protect the dopaminergic neurons of the rats suffering Parkinsonism. Besides, increasing the TH+ levels of the brain tissue of the rats can relieve dystonia caused by deficiency of tyrosine hydroxylase.

### Embodiment VIII: Analysis of Activities of Mitochondria of Brain Tissue and Muscle Tissue

Some researches indicate that the dysfunction of the mitochondria of brain tissue correlate with the degeneration of the dopaminergic neurons. The mitochondria are especially active in brain cells, participating in many important biological processes, including regulation of free radicals and neuro-transmitters. In fact, monoamine oxidase (MAO), which is responsible for metabolism of monoamine neuro-transmitters, is exactly in the outer membranes of the mitochondria. The dysfunction of mitochondria will decrease the energy generated by adenosine triphosphate (ATP) and raise oxidative stress. Oxidative stress is likely to be found in the brains of the patients suffering brain diseases and mental diseases. It is also indicated by some researches: the mitochondria of the patients suffering dementia are less active and unable to supply sufficient energy to the brains.

In the experiment, after the rats are sacrificed, brain tissues and muscle tissues are taken out and placed in dishes having a culture medium containing Dulbecco's modified minimal essential (DMEM), wherein DMEM includes 2% Fetal Bovine Serum (FBS). After the tissue is taken out, the tissue is placed in a glass tissue homogenizer containing a monoammonium succinate (MAS) solution and ground to a completely broken state. Next, centrifuge the broken tissue and remove the supernatant thereof. Next, add a BSA-free MSA solution to the broken tissue, centrifuge it, and remove the supernatant thereof. Next, add 1 mL BSA-free MSA solution to the underneath deposition and mix them. Then, take out the mitochondria-containing solution, and quantitate protein.

The tissue culture dish is analyzed with the XF24 extracellular flux analyzer. This instrument uses an optical probe to detect the sample, not using any chemical reagent, not contacting the sample, and not damaging the cells. In detection, the probe system approaches the cells and forms a temporary enclosed space in the bottom of the culture dish. The instrument detects the oxygen consumption rate and the acid generation rate in the tiny space to instantly observe the operation of the mitochondria in cells. The analyzing instrument is integrated with an automatic drug injection system. The following drugs are added in sequence: oligomycin (mitochondria ATP synthase inhibitor), Carbonyl cyanide 4-(trifluoromethoxy) phenylhydrazone (FCCP), antimycin A, the mitochondria complex III inhibitor (drilling holes in the membrane of mitochondria) so as to observe the basal respiration, ATP production, maximal respiration, membrane integrity (proton leak) of mitochondria.

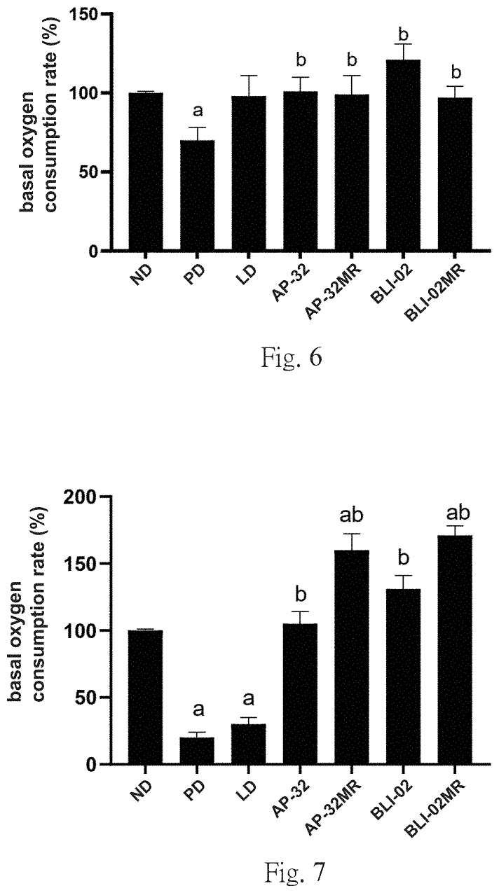

The results of the analysis of the activity of mitochondria in the brain tissues of the rats are shown in FIG. 6, and the results of the analysis of the activity of mitochondria in the muscle tissues of the rats are shown in FIG. 7, wherein the symbol a indicates that the result has significant difference with respect to Group ND, and p<0.05; the symbol b indicates that the result has significant difference with respect to Group PD, and p<0.05. The statistical analysis uses One-way ANOVA, tested with Tukey HSD, and expressed as mean±SEM. Refer to FIG. 6. The mitochondria of the brain tissues of the rats in the following groups have the best respiration performance (basal oxygen consumption rate), including Group AP-32 whose rats are fed with the AP-32 strain of Lactobacillus salivarius subsp. salicinius for 14 weeks, Group BLI-02 whose rats are fed with the BLI-02 strain of Bifidobacterium longum subsp. infantis for 14 weeks, Group AP-32MR whose rats are fed with the fermentation metabolites of the AP-32 strain of Lactobacillus salivarius subsp. salicinius for 14 weeks and Group BLI-02MR whose rats are fed with the fermentation metabolites of the BLI-02 strain of Bifidobacterium longum subsp. infantis for 14 weeks. These groups have significant difference with respect to Group PD. Refer to FIG. 7. The mitochondria of the muscle tissues of the rats in the following groups have the best respiration performance (basal oxygen consumption rate), including Group AP-32 whose rats are fed with the AP-32 strain of Lactobacillus salivarius subsp. salicinius for 14 weeks, Group BLI-02 whose rats are fed with the BLI-02 strain of Bifidobacterium longum subsp. infantis for 14 weeks, Group AP-32MR whose rats are fed with the fermentation metabolites of the AP-32 strain of Lactobacillus salivarius subsp. salicinius for 14 weeks and Group BLI-02MR whose rats are fed with the fermentation metabolites of the BLI-02 strain of Bifidobacterium longum subsp. infantis for 14 weeks. These groups have significant difference with respect to Group PD. Especially, the groups whose rats are fed with the fermentation metabolites of lactic acid bacterium strains of the present invention (Group AP-32MR and Group BLI-02MR) have further better respiration performance. Feeding the rats with the AP-32 strain of Lactobacillus salivarius subsp. salicinius, the BLI-02 strain of Bifidobacterium longum subsp. infantis or the fermentation metabolites thereof can effectively increase the ratio of mitochondria in the brain tissues of the rats. It indicates that the AP-32 strain of Lactobacillus salivarius subsp. salicinius, the BLI-02 strain of Bifidobacterium longum subsp. infantis and the fermentation metabolites thereof are favorable to relieve dementia or physical stress, which is induced by dysfunctional mitochondria. Some documents point out that Parkinsonism may be accompanied by weight loss and muscle loss. The ratio of mitochondria is increased in the rats fed with the AP-32 strain of Lactobacillus salivarius subsp. salicinius, the BLI-02 strain of Bifidobacterium longum subsp. infantis or the fermentation metabolites thereof. Enhancing the activity of mitochondria in muscle can raise the energy utilization rate of the muscle. Therefore, the present invention is favorable to recover body weight and muscle mass and improve the performance in muscle exercise.

### Embodiment IX: Analysis of Effect of Lactic Acid Bacterium Strains on Protecting Nerve Cells

In the paper by Park et al., the oxidizing substance H₂O₂ is added to the SH-SY5Y strain of neuroblastoma cells to cause damage and death of the SH-SY5Y strain; and the cell model is used to establish an in-vitro drug screening platform of Parkinsonism. (Park et al., (2017). “Neuroprotective effect of Ruminococcus albus on oxidatively stressed SH-SY5Y cells and animals”. Nature, 7:14520. https://doi.org/10.1038/s41598-017-15163-5)

The experiment includes two stages. In the first stage, cultivate the strain of live lactic acid bacteria and the Caco-2 intestinal cells together and collect the supernatant thereof to simulate the situation that the lactic acid bacterium strain colonizes in the intestinal tract and secretes beneficial materials in the human body. In the second stage, add the supernatant, which is generated by the co-culture of the live bacterium strain and the Caco-2 intestinal cells, into the SH-SY5Y strain; further add the oxidizing substance H₂O₂ to the SH-SY5Y strain to cause damage and death of the SH-SY5Y strain. The effects of the secretions of different groups of lactic acid bacterium strains on protecting nerve cells of brains are learned via calculating the survival rates of nerve cells in different groups.

The detailed process of the first stage is as follows: cultivate the Caco-2 cells of each group in the wells of a 6-well culture plate, and place the 6-well culture plate in an incubator with 5% CO2 at a temperature of 37° C., wherein 6×10⁵ cells are cultivated in each well, and wherein each well contains 3 ml DMEM culture medium, and wherein the DMEM culture medium 10% Fetal Bovine Serum (FBS). After 10 days' cultivation, the Caco-2 cells agglomerate to form a cellular monolayer. Next, add each group of probiotic to the Caco-2 cells by a density of 1×10⁷ CFU/mL, and cultivate them for 24 hours. The experiment groups, whose probiotics are added into the Caco-2 cells, include

(1) Group (AP-32) using the AP-32 strain of Lactobacillus salivarius subsp. salicinius;
(2) Group (BLI-02) using the BLI-02 strain of Bifidobacterium longum subsp. infantis;
(3) Group (L-28) using the L-28 strain of L. salivarius;
(4) Group (L-55) using the L-55 strain of B. infantis;
(5) Group (LGG) using the LGG strain of Lactobacillus rhamnosus (Chr. Hansen, Hoersholm, Denmark);
(6) Group (BB-12) using the BB-12 strain of Bifidobacterium animalis subsp. lactis (Chr. Hansen, Hoersholm, Denmark);
(7) Group (1:9) using a mixture of AP-32:BLI-02 by a ratio of 1:9;
(8) Group (1:8) using a mixture of AP-32:BLI-02 by a ratio of 1:8;
(9) Group (1:1) using a mixture of AP-32:BLI-02 by a ratio of 1:1;
(10) Group (8:1) using a mixture of AP-32:BLI-02 by a ratio of 8:1;
(11) Group (9:1) using a mixture of AP-32:BLI-02 by a ratio of 9:1;
(12) Group (AP-32+L-55) using a mixture of AP-32:L-55 by a ratio of 1:1;
(13) Group (BLI-02+L-28) using a mixture of BLI-02:L-28 by a ratio of 1:1; and
(14) Group (L-28+L-55) using a mixture of L-28:L-55 by a ratio of 1:1.

After 24 hour cultivation, use a centrifugal force of 14000×g to centrifuge the cultivation product for 5 minutes to collect the supernatant of each group. Next, use a filter with a pore size of 0.45 μm to filter the supernatant. In all the experiments, the Caco-2 cells prepared with the DMEM culture medium are used as the blank control groups.

In the second stage, the oxidizing material H₂O₂ is used to generate the oxidative stress model of cells, whereby to evaluate the effect of the fermentation liquids on protecting the SH-SY5Y strain. The detailed process of the second stage is as follows: grow the SH-SY5Y strain in a high-glucose DMEM culture medium in an incubator with 5% CO2 and 95% air and at a temperature of 37° C. The number of the subculture cycles of the SH-SY5Y strain is less than 30. Each group of cells (1×10⁵ cells/well) is planted into a 96-well plate. Next, add 100 μl supernatant to each well, wherein the supernatant is prepared in the first stage (Experiment Groups (1)-(14)). In the control group, only the oxidizing material and the DMEM culture medium is added to the SH-SY5Y strain, and no bacterium liquid is added. Each group of bacterium liquid is cultivated together with the SH-SY5Y strain for 4 hours. The oxidizing material H₂O₂ is used to induce oxidation so as to evaluate the effect of the bacterium liquid on protecting the SH-SY5Y strain. Each group of cells (1×10⁵ cells/well) is cultivated together with the oxidizing material H₂O₂ (50 μM) in a 96-well plate for 20 hours. Next, remove the culture medium, and cultivate the cells together with 100 μL MTT for 1 hour. Next, use a spectrophotometer to measure the light absorbance at the wavelength of 570 nm. The survival rate of nerve cells is the percentage with respect to the cells of the blank control group.

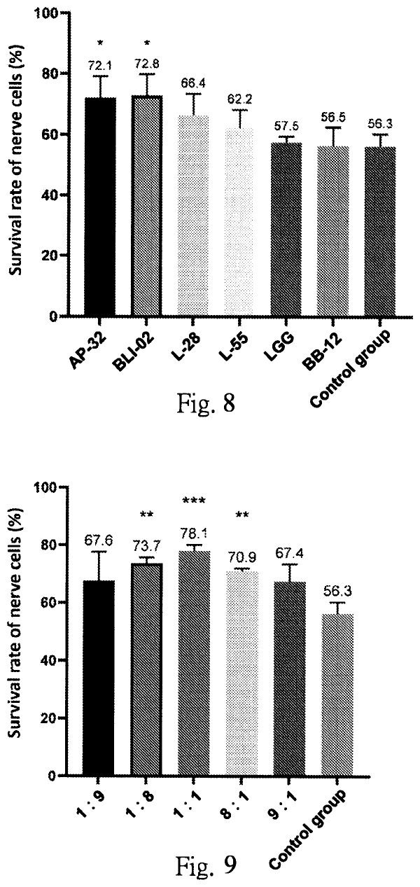

FIG. 8 shows the experiment results of the effects of single lactic acid bacterium strains on protecting nerve cells, wherein * indicates that the result has significant difference with respect to the control group and p<0.05. FIG. 8 shows that the AP-32 strain of Lactobacillus salivarius subsp. salicinius and the BLI-02 strain of Bifidobacterium longum subsp. infantis of the present invention have superior ability of protecting nerve cells in comparison with the control group and that the difference between Group (AP-32)/Group (BLI-02) and the control group is significant. The nerve cell protection ability of the AP-32 strain of Lactobacillus salivarius subsp. salicinius and the BLI-02 strain of Bifidobacterium longum subsp. infantis of the present invention is also better than that of the L-28 strain of L. salivarius, the L-55 strain of B. infantis, the LGG strain of Lactobacillus rhamnosus, and the BB-12 strain of Bifidobacterium animalis subsp. lactis.

FIG. 9 shows the experiment results of the nerve cell protection effects of the mixtures of lactic acid bacterium strains of the present invention by different ratios, wherein ** indicates that the result has significant difference with respect to the control group and p<0.01, and wherein *** indicates that the result has significant difference with respect to the control group and p<0.001. FIG. 9 shows that the mixtures of the AP-32 strain of Lactobacillus salivarius subsp. salicinius and the BLI-02 strain of Bifidobacterium longum subsp. infantis of the present invention by the ratios of from 1:8 to 8:1 have superior ability of protecting nerve cells in comparison with the control group and that the mixtures of the AP-32 strain and the BLI-02 strain by the ratios of from 1:8 to 8:1 have significant difference with respect to the control group. Especially, the mixture of the AP-32 strain and the BLI-02 strain by the ratio of 1:1 is further superior to the other mixtures. In comparison with the experiment results in FIG. 8, the differences between the mixtures of the AP-32 strain of Lactobacillus salivarius subsp. salicinius and the BLI-02 strain of Bifidobacterium longum subsp. infantis of the present invention by the ratios of from 1:8 to 8:1 and the control group have higher confidence level.

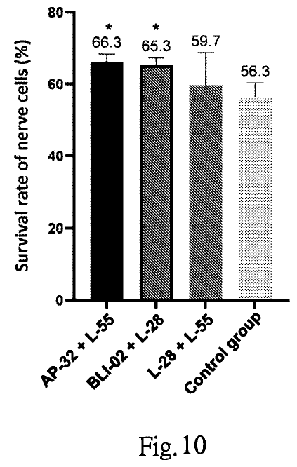

FIG. 10 shows the experiment results of the nerve cell protection effects of the mixtures where the lactic acid bacterium strain of the present invention is mixed with a different strain of the same genus and the same species by a ratio of 1:1, wherein * indicates that the result has significant difference with respect to the control group and p<0.05. The mixtures include a mixture of the AP-32 strain of Lactobacillus salivarius subsp. salicinius of the present invention and the L-28 strain of the same genus and the same species, a mixture of the BLI-02 strain of Bifidobacterium longum subsp. infantis of the present invention and the L-55 strain of the same genus and the same species, and a mixture of the L-28 strain of the same genus and the same species of the AP-32 strain and the L-55 strain of the same genus and the same species of the BLI-02 strain. FIG. 10 shows that the mixture of the AP-32 strain and the L-28 strain by the ratio of 1:1 and the mixture of the BLI-02 strain and the L-55 strain of the ratio of 1:1 have the nerve cell protection effects and that the abovementioned mixtures have significant difference with respect to the control group. It should be noted: the 1:1 mixture of the L-28 strain and the L-55 strain, which are respectively of the same genera and the same species of the AP-32 strain and the BLI-02 strain, do not have significant difference with respect to the control group. Therefore, the AP-32 strain of Lactobacillus salivarius subsp. salicinius and BLI-02 strain of Bifidobacterium longum subsp. infantis of the present invention have nerve cell protection ability specifically.

In conclusion, the present invention provides a composition containing the AP-32 strain of Lactobacillus salivarius subsp. salicinius, the BLI-02 strain of Bifidobacterium longum subsp. infantis or the fermentation metabolites thereof, which has the physiological activity of elevating the ability of brain tissue. The present invention may be in form of a food composition or a pharmaceutical composition to elevate the ability of brain tissue.

While the invention is susceptible to various modifications and alternative forms, a specific example thereof has been shown in the drawings and is herein described in detail. It should be understood, however, that the invention is not to be limited to the particular form disclosed, but to the contrary, the invention is to cover all modifications, equivalents, and alternatives falling within the appended claims.

### Bioresource Deposition

CCTCC NO: M2011127, China Center for Type Culture Collection, Apr. 10, 2011

CGMCC No. 15212, China General Microbiological Culture Collection Center, Jan. 15, 2018

What is claimed is:

1. An application of using at least one of lactic acid bacterium strains and fermentation metabolites thereof to fabricate a food composition for elevating ability of brain tissue, wherein the food composition comprises
at least one of isolated lactic acid bacterium strains and the fermentation metabolites thereof, wherein the isolated lactic acid bacterium strain is selected from a group including an AP-32 strain of Lactobacillus salivarius subsp. salicinius with a deposition number of CCTCC NO: M2011127, a BLI-02 strain of Bifidobacterium longum subsp. infantis with a deposition number of CGMCC No. 15212, and combinations thereof, and wherein the abovementioned strains are respectively deposited in China Center for Type Culture Collection (CCTCC) and China General Microbiological Culture Collection Center (CGMCC); and
a physiologically-acceptable excipient, diluent or carrier.

2. The application of using at least one of lactic acid bacterium strains and fermentation metabolites thereof to fabricate a food composition for elevating ability of brain tissue according to claim 1, wherein the at least one of isolated lactic acid bacterium strains is a mixture of the AP-32 strain of Lactobacillus salivarius subsp. salicinius and the BLI-02 strain of Bifidobacterium longum subsp. infantis by a ratio of from 1:8 to 8:1.

3. The application of using at least one of lactic acid bacterium strains and fermentation metabolites thereof to fabricate a food composition for elevating ability of brain tissue according to claim 1, wherein the at least one of isolated lactic acid bacterium strains is a mixture of the AP-32 strain of Lactobacillus salivarius subsp. salicinius and the BLI-02 strain of Bifidobacterium longum subsp. infantis by a ratio of 1:1.

4. The application of using at least one of lactic acid bacterium strains and fermentation metabolites thereof to fabricate a food composition for elevating ability of brain tissue according to claim 1, wherein the lactic acid bacterium strain is an active strain.

5. The application of using at least one of lactic acid bacterium strains and fermentation metabolites thereof to fabricate a food composition for elevating ability of brain tissue according to claim 1, wherein the fermentation metabolite includes deactivated strains, a supernatant of a fermentation liquid where bacterium cells are removed, a whey of a fermentation liquid, or a dried powder of the abovementioned deactivated strains, supernatant or whey.

6. The application of using at least one of lactic acid bacterium strains and fermentation metabolites thereof to fabricate a food composition for elevating ability of brain tissue according to claim 1, wherein the physiologically-acceptable excipient, diluent or carrier is a food.

7. The application of using at least one of lactic acid bacterium strains and fermentation metabolites thereof to fabricate a food composition for elevating ability of brain tissue according to claim 1, wherein the physiologically-acceptable excipient, diluent or carrier is fermented milk, yoghurt, cheese, powdered milk, tea, coffee, candy, functional beverages or a combination thereof.

8. The application of using at least one of lactic acid bacterium strains and fermentation metabolites thereof to fabricate a food composition for elevating ability of brain tissue according to claim 1, wherein the lactic acid bacterium strain is cultivated in a culture medium; the culture medium contains at least one of carbon sources and nitrogen sources; the carbon source includes glucose, fructose, lactose, sucrose, maltose, galactose, mannose, trehalose, starch, molasses, potato starch, maize starch, malt extract, maltodextrin, or a combination thereof; the nitrogen source includes (NH₄)₂SO₄, (NH₄)₃PO₄, NH₄NO₃, NH₄Cl, casamino acid, urea, peptone, polypeptone, tryptone, meat extract, yeast extract, yeast powder, milk, soybean flour, whey, or a combination thereof.

9. The application of using at least one of lactic acid bacterium strains and fermentation metabolites thereof to fabricate a food composition for elevating ability of brain tissue according to claim 1, wherein the lactic acid bacterium strain is cultivated in a culture medium; the culture medium includes 2-5 wt. % of a mixture of glucose and maltodextrin.

10. The application of using at least one of lactic acid bacterium strains and fermentation metabolites thereof to fabricate a food composition for elevating ability of brain tissue according to claim 1, wherein the lactic acid bacterium strain is cultivated in a liquid culture medium; the liquid culture medium includes at least one of 5-30 wt. % milk and 1-10 wt. % soybean flour.

11. An application of using at least one of lactic acid bacterium strains and fermentation metabolites thereof to fabricate a pharmaceutical composition for elevating ability of brain tissue, wherein the pharmaceutical composition comprises
at least one of isolated lactic acid bacterium strains and the fermentation metabolites thereof, wherein the isolated lactic acid bacterium strain is selected from a group including an AP-32 strain of Lactobacillus salivarius subsp. salicinius with a deposition number of CCTCC NO: M2011127, a BLI-02 strain of Bifidobacterium longum subsp. infantis with a deposition number of CGMCC No. 15212, and combinations thereof, and wherein the abovementioned strains are respectively deposited in China Center for Type Culture Collection (CCTCC) and China General Microbiological Culture Collection Center (CGMCC); and
a pharmaceutically-acceptable excipient, diluent or carrier.

12. The application of using at least one of lactic acid bacterium strains and fermentation metabolites thereof to fabricate a pharmaceutical composition for elevating ability of brain tissue according to claim 11, wherein the at least one of isolated lactic acid bacterium strains is a mixture of the AP-32 strain of Lactobacillus salivarius subsp. salicinius and the BLI-02 strain of Bifidobacterium longum subsp. infantis by a ratio of from 1:8 to 8:1.

13. The application of using at least one of lactic acid bacterium strains and fermentation metabolites thereof to fabricate a pharmaceutical composition for elevating ability of brain tissue according to claim 11, wherein the at least one of isolated lactic acid bacterium strains is a mixture of the AP-32 strain of Lactobacillus salivarius subsp. salicinius and the BLI-02 strain of Bifidobacterium longum subsp. infantis by a ratio of 1:1.

14. The application of using at least one of lactic acid bacterium strains and fermentation metabolites thereof to fabricate a pharmaceutical composition for elevating ability of brain tissue according to claim 11, wherein the lactic acid bacterium strain is an active strain.

15. The application of using at least one of lactic acid bacterium strains and fermentation metabolites thereof to fabricate a pharmaceutical composition for elevating ability of brain tissue according to claim 11, wherein the fermentation metabolite includes deactivated strains, a supernatant of a fermentation liquid where bacterium cells are removed, a whey of a fermentation liquid, or a dried powder of the abovementioned deactivated strains, supernatant or whey.

16. The application of using at least one of lactic acid bacterium strains and fermentation metabolites thereof to fabricate a pharmaceutical composition for elevating ability of brain tissue according to claim 11, wherein the pharmaceutical composition is in form of an oral dosage.

17. The application of using at least one of lactic acid bacterium strains and fermentation metabolites thereof to fabricate a pharmaceutical composition for elevating ability of brain tissue according to claim 11, wherein the pharmaceutical composition is in form of a tablet, a capsule, a solution, or a powder.

18. The application of using at least one of lactic acid bacterium strains and fermentation metabolites thereof to fabricate a pharmaceutical composition for elevating ability of brain tissue according to claim 11, wherein the lactic acid bacterium strain is cultivated in a culture medium; the culture medium contains at least one of carbon sources and nitrogen sources; the carbon source includes glucose, fructose, lactose, sucrose, maltose, galactose, mannose, trehalose, starch, molasses, potato starch, maize starch, malt extract, maltodextrin, or a combination thereof; the nitrogen source includes (NH₄)₂SO₄, (NH₄)₃PO₄, NH₄NO₃, NH₄Cl, casamino acid, urea, peptone, polypeptone, tryptone, meat extract, yeast extract, yeast powder, milk, soybean flour, whey, or a combination thereof.

19. The application of using at least one of lactic acid bacterium strains and fermentation metabolites thereof to fabricate a pharmaceutical composition for elevating ability of brain tissue according to claim 11, wherein the lactic acid bacterium strain is cultivated in a culture medium; the culture medium includes 2-5 wt. % of a mixture of glucose and maltodextrin.

20. The application of using at least one of lactic acid bacterium strains and fermentation metabolites thereof to fabricate a pharmaceutical composition for elevating ability of brain tissue according to claim 11, wherein the lactic acid bacterium strain is cultivated in a liquid culture medium; the liquid culture medium includes at least one of 5-30 wt. % milk and 1-10 wt. % soybean flour.

21. A composition containing at least one of lactic acid bacterium strains and fermentation metabolites thereof, which comprises
at least one of isolated lactic acid bacterium strains and the fermentation metabolites thereof, wherein the lactic acid bacterium strains have a physiological activity of elevating ability of brain tissue, and wherein the isolated lactic acid bacterium strain is selected from a group including an AP-32 strain of Lactobacillus salivarius subsp. salicinius with a deposition number of CCTCC NO: M2011127, a BLI-02 strain of Bifidobacterium longum subsp. infantis with a deposition number of CGMCC No. 15212, and combinations thereof, and wherein the abovementioned strains are respectively deposited in China Center for Type Culture Collection (CCTCC) and China General Microbiological Culture Collection Center (CGMCC); and
a physiologically or pharmaceutically-acceptable excipient, diluent, or carrier.

22. The composition containing at least one of lactic acid bacterium strains and fermentation metabolites thereof according to claim 21, wherein the at least one of isolated lactic acid bacterium strains is a mixture of the AP-32 strain of Lactobacillus salivarius subsp. salicinius and the BLI-02 strain of Bifidobacterium longum subsp. infantis by a ratio of from 1:8 to 8:1.

23. The composition containing at least one of lactic acid bacterium strains and fermentation metabolites thereof according to claim 21, wherein the at least one of isolated lactic acid bacterium strains is a mixture of the AP-32 strain of Lactobacillus salivarius subsp. salicinius and the BLI-02 strain of Bifidobacterium longum subsp. infantis by a ratio of 1:1.

24. The composition containing at least one of lactic acid bacterium strains and fermentation metabolites thereof according to claim 21, wherein the lactic acid bacterium strain is an active strain.

25. The composition containing at least one of lactic acid bacterium strains and fermentation metabolites thereof according to claim 21, wherein the fermentation metabolite includes deactivated strains, a supernatant of a fermentation liquid where bacterium cells are removed, a whey of a fermentation liquid, or a dried powder of the abovementioned deactivated strains, supernatant or whey.

26. The composition containing at least one of lactic acid bacterium strains and fermentation metabolites thereof according to claim 21, wherein the recipient, diluent or carrier is a food.

27. The composition containing at least one of lactic acid bacterium strains and fermentation metabolites thereof according to claim 21, wherein the recipient, diluent or carrier is fermented milk, yoghurt, cheese, powdered milk, tea, coffee, candy, functional beverages or a combination thereof.

28. The composition containing at least one of lactic acid bacterium strains and fermentation metabolites thereof according to claim 21, which is a pharmaceutical composition in form of an oral dosage.

29. The composition containing at least one of lactic acid bacterium strains and fermentation metabolites thereof according to claim 21, which is a pharmaceutical composition in form of an oral dosage, wherein the oral dosage is in form of a tablet, a capsule, a solution, or a powder.

30. The composition containing at least one of lactic acid bacterium strains and fermentation metabolites thereof according to claim 21, wherein the lactic acid bacterium strain is cultivated in a culture medium; the culture medium contains at least one of carbon sources and nitrogen sources; the carbon source includes glucose, fructose, lactose, sucrose, maltose, galactose, mannose, trehalose, starch, molasses, potato starch, maize starch, malt extract, maltodextrin, or a combination thereof; the nitrogen source includes (NH₄)₂SO₄, (NH₄)₃PO₄, NH₄NO₃, NH₄Cl, casamino acid, urea, peptone, polypeptone, tryptone, meat extract, yeast extract, yeast powder, milk, soybean flour, whey, or a combination thereof.

31. The composition containing at least one of lactic acid bacterium strains and fermentation metabolites thereof according to claim 21, wherein the lactic acid bacterium strain is cultivated in a culture medium; the culture medium includes 2-5 wt. % of a mixture of glucose and maltodextrin.

32. The composition containing at least one of lactic acid bacterium strains and fermentation metabolites thereof according to claim 21, wherein the lactic acid bacterium strain is cultivated in a liquid culture medium; the liquid culture medium includes at least one of 5-30 wt. % milk and 1-10 wt. % soybean flour.
